(19) United States
(12) Patent Application Publication
(10) Pub. No.: US 2009/0170878 A1
(43) Pub. Date: Jul. 2, 2009

(54) MACROCYCLIC COMPOUNDS USEFUL AS BACE INHIBITORS

(76) Inventor: Rainer Machauer, Freiburg (DE)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/374,469

(22) PCT Filed: Jul. 19, 2007
(86) PCT No.: PCT/EP2007/057492

§ 371 Date: Jan. 20, 2009

(30) Foreign Application Priority Data
Jul. 20, 2006 (EP) 06117583.2

(51) Int. Cl. A61K 31/519; C07D 225/02; A61K 31/4353; A61K 31/33; C07D 487/00; A61P 25/00; A61P 9/00

(52) U.S. Cl. 514/258.1; 540/451; 514/300; 514/183; 540/461

(57) ABSTRACT

The invention relates to novel macrocyclic compounds of the formula

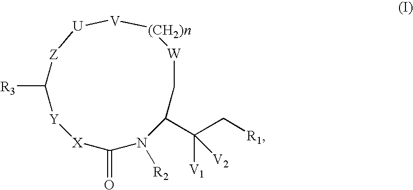

in which all of the variables are as defined in the specification, in free base form or in acid addition salt form, to their preparation, to their use as medicaments and to medicaments comprising them.

The present invention relates to novel macrocyclic compounds, to their preparation, to their use as medicaments and to medicaments comprising them.

More particularly, the invention relates to a compound of the formula

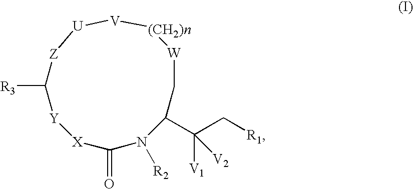

in which
R₁ is —(CH₂)ₖN(Rₐ)R_{b}, in which
   k is 0, 1 or 2;
   Rₐ is hydrogen or an optionally substituted (C₁₋₈)alkyl, (C₃₋₈)cycloalkyl, (C₃₋₈)cycloalkyl-(C₁₋₄)alkyl, aryl, aryl(C₁₋₄)alkyl, heteroaryl, heteroaryl(C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydro-quinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]oxathiin-4-yl, 2,3,4,5-tetrahydro-benzo[b]oxepin-5-yl or 1,3,4,5-tetrahydro-benzo[c]oxepin-5-yl group; and
   R_{b} is a (C₃₋₈)cycloalkyl group, in which
      (a) one of the carbon ring members of the (C₃₋₈)cycloalkyl moiety, which are different from the carbon ring member, to which the nitrogen atom carrying Rₐ is attached, is optionally replaced by a hetero ring member, selected from the group consisting of —O—, —S—, —S(═O)—, —S(═O)₂— and —N(R_{c})—, in which
         R_{c} is hydrogen or an optionally substituted (C₁₋₈)alkyl, (C₃₋₈)cycloalkyl, (C₃₋₈)cycloalkyl(C₁₋₄)alkyl, aryl, aryl(C₁₋₄)alkyl, heteroaryl or heteroaryl(C₁₋₄)alkyl group,
      (b) the (C₃₋₈)cycloalkyl moiety is substituted by 1 to 4 substituents, independently selected from the group consisting of halogen, cyano, oxo, hydroxy, (C₁₋₄)-alkoxy, (C₁₋₄)alkoxy(C₁₋₄)alkoxy, (C₁₋₄)alkylthio, (C₁₋₄)alkylsulfinyl, (C₁₋₄)alkylsulfonyl, (C₁₋₄)alkylcarbonyl, (C₁₋₄)alkylcarbonyloxy, (C₁₋₄)alkoxycarbonyl, (C₁₋₄)alkoxycarbonyloxy and an optionally substituted (C₁₋₈)alkyl, (C₃₋₈)cycloalkyl, (C₃₋₈)cycloalkyl-(C₁₋₄)alkyl, aryl, aryl(C₁₋₄)alkyl, heteroaryl, heteroaryl(C₁₋₄)alkyl, non-aromatic heterocyclyl, non-aromatic heterocyclyl(C₁₋₄)alkyl, chroman-4-yl, isochroman-4-yl, thiochroman-4-yl, isothiochroman-4-yl, 1,1-dioxo-1 ambda*6*-thiochroman-4-yl, 2,2-dioxo-2lambda*6*-isothiochroman-4-yl, 1,2,3,4-tetrahydro-quinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]oxathiin-4-yl, 2,3,4,5-tetrahydro-benzo[b]oxepin-5-yl or 1,3,4,5-tetrahydro-benzo[c]-oxepin-5-yl group, and
      (c) the (C₃₋₈)cycloalkyl moiety is optionally substituted at two adjacent carbon ring members by two substituents, which form, together with the two adjacent carbon ring members, to which they are attached, a (C₃₋₈)cycloalkyl group, in which
         (i) one of the carbon ring members of the (C₃₋₈)cycloalkyl group thus formed, which are different from the said two adjacent carbon ring members, to which the said two substituents are optionally attached, is optionally replaced by a hetero ring member, selected from the group consisting of —O—, —S—, —S(═O)—, —S(═O)₂— and —N(R_{d})—, in which
            R_{d} is hydrogen or an optionally substituted (C₁₋₈)alkyl, (C₃₋₈)cycloalkyl, (C₃₋₈)cycloalkyl(C₁₋₄)alkyl, aryl, aryl(C₁₋₄)alkyl, heteroaryl or heteroaryl(C₁₋₄)alkyl group, and
         (ii) the (C₃₋₈)cycloalkyl group thus formed is optionally substituted by 1 to 4 substituents, independently selected from the group consisting of halogen, cyano, oxo, hydroxy, (C₁₋₄)alkoxy, (C₁₋₄)alkoxy(C₁₋₄)alkoxy, (C₁₋₄)alkylthio, (C₁₋₄)alkylsulfinyl, (C₁₋₄)alkylsulfonyl, (C₁₋₄)alkylcarbonyl, (C₁₋₄)alkylcarbonyloxy, (C₁₋₄)alkoxycarbonyl, (C₁₋₄)alkoxycarbonyloxy and an optionally substituted (C₁₋₈)alkyl, (C₃₋₈)cycloalkyl, (C₃₋₈)cycloalkyl(C₁₋₄)alkyl, aryl, aryl(C₁₋₄)alkyl, heteroaryl, heteroaryl(C₁₋₄)alkyl, non-aromatic heterocyclyl, non-aromatic heterocyclyl(C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydro-quinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]-oxathiin-4-yl, 2,3,4,5-tetrahydro-benzo[b]oxepin-5-yl or 1,3,4,5-tetrahydro-benzo[c]-oxepin-5-yl group;
R₂ is hydrogen or (C₁₋₈)alkyl;
R₃ is hydrogen, (C₁₋₈)alkyl or an optionally substituted (C₁₋₈)alkylOC(═O)NH, (C₃₋₈)cycloalkylOC(═O)NH, (C₃₋₈)cycloalkyl(C₁₋₄)alkylOC(═O)NH, aryl(C₁₋₄)alkylOC(═O)NH, heteroaryl(C₁₋₄)alkylOC(═O)NH, (C₁₋₄)alkylC(═O)NH, (C₃₋₈)cycloalkylC(═O)NH, arylC(═O)NH, aryl(C₁₋₄)alkylC(═O)NH, heteroarylC(═O)NH or heteroaryl(C₁₋₄)alkylC(═O)NH group;
U is a bond, CF₂, CF₂CF₂, CHF, CHFCHF, cycloprop-1,2-ylene, (C₁₋₃)alkylenoxy, (C₁₋₃)alkylenamino, (C₁₋₈)alkylene, NRₑ or an aromatic or heteroaromatic ring, which ring is optionally substituted with halogen, (C₁₋₈)alkoxy, hydroxy or (C₁₋₈)alkyl, whereby Z and V are in ortho- or meta-position to each other, wherein
   Rₑ is hydrogen, (C₁₋₈)alkyl or (C₃₋₇)cycloalkyl;
V is CH═CH, cycloprop-1,2-ylene, CH₂CH(OH), CH(OH)CH₂ or CR_{f}R_{f}CR_{f}R_{f}, wherein
   each R_{f}, independently, is hydrogen, fluorine or (C₁₋₈)alkyl;
      either
V₁ is hydrogen and
V₂ is hydroxy
   or
V₁ and V₂ together are oxo;
W is (C₁₋₈)alkylene, O, S, S(═O)₂, C(═O), C(═O)O, OC(═O), N(R_{g})C(═O), C(═O)NR_{g} or NR_{g}, wherein
   R_{g} is hydrogen or (C₁₋₈)alkyl;
X is optionally substituted (C₁₋₈)alkylene or an optionally substituted (C₃₋₈)cycloalkylene, piperidinediyl or pyrrolidinediyl group, to which group Y and C(═O)NR₂ are attached in meta-position to each other;
Y is a bond, O, S(═O)₂, S(═O)₂NRₕ, N(Rₕ)S(═O)₂, NRₕ, C(Rₕ)OH, C(═O)NRₕ, N(Rₕ)C(═O), C(═O)N(Rₕ)O or ON(Rₕ)C(═O), wherein
   Rₕ is hydrogen, (C₁₋₈)alkyl or (C₃₋₈)cycloalkyl;
Z is O, CH₂, CF₂, CHF, CH═CH, cycloprop-1,2-ylene or a bond; and
n is 0 to 5,
   the number of ring atoms included in the macrocyclic ring being 14, 15, 16 or 17, in free base form or in acid addition salt form.

E. g. on account of one or more than one asymmetrical carbon atom, which may be present in a compound of the formula I, a corresponding compound of the formula I may exist in pure optically active form or in the form of a mixture of optical isomers, e. g. in the form of a racemic mixture. All of such pure optical isomers and all of their mixtures, including the racemic mixtures, are part of the present invention.

A compound of the formula I may exist in free base form or in acid addition salt form. All of such free compounds and salts are part of the present invention.

A compound of the formula I may exist in tautomeric form. All of such tautomers are part of the present invention.

Halogen denotes fluorine, chlorine, bromine or iodine.

Optional substituents on alkyl, cycloalkyl or non-aromatic heterocyclyl groups or moieties may be one to four groups independently selected from hydroxy, hydroxy(C₁₋₄)alkyl, (C₁₋₄)-alkoxy, (C₁₋₄)alkoxy(C₁₋₄)alkyl, (C₁₋₄)alkoxy(C₁₋₄)alkoxy, (C₁₋₄)alkylsulfanyl, (C₁₋₄)alkoxycarbonyl, (C₁₋₄)alkylcarbonyloxy, (C₁₋₄)alkylcarbonyl, (C₁₋₄)alkylsulfonyl, cyano, oxo, (C₃₋₇)cycloalkyl, optionally substituted aryl, optionally substituted aryl(C₁₋₄)alkyl, optionally substituted heteroaryl and optionally substituted heteroaryl(C₁₋₄)alkyl.

Optional substituents on chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydroquinol-4-yl, 1,2,3,4-tetrahydroisoquinol-4-yl, 1,2,3,4-tetrahydronaphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c]-[1,2]oxathiin-4-yl, 2,2-dioxo-3,4-dihydro-2H-2lambda*6*-benzo[e][1,2]oxathiin-4-yl, 2,3,4,5-tetrahydrobenzo[b]oxepin-5-yl, 1,3,4,5-tetrahydrobenzo[c]oxepin-5-yl, aryl or heteroaryl groups or moieties may be one to four, especially one to three, groups independently selected from hydroxy, (C₁₋₈)alkyl, (C₁₋₆)alkoxy, (C₁₋₄)alkoxy(C₁₋₄)alkyl, S(═O)₂(C₁₋₄)alkyl, (C₃₋₇)-cycloalkyl, (C₃₋₇)cycloalkyl(C₁₋₄)alkyl, cyano, nitro, trifluoromethyl, halogen, optionally substituted aryl, optionally substituted heteroaryl and optionally substituted carbamoyl.

An optionally substituted aryl or heteroaryl group or moiety may also carry, as optional substituents, one to three groups selected from benzyloxy, phenoxy, S(═O)₂NH₂, N(H)S(═O)₂(C₁₋₃)alkyl, carboxy, (C₁₋₄)alkoxycarbonyl, (C₁₋₄)alkylcarbamoyl, (C₁₋₄)alkylcarbonyloxy, (C₁₋₄)alkylcarbonyl, hydroxy(C₁₋₄)alkyl and optionally substituted amino.

Optional substituents on alkylene, cycloalkylene, piperidinediyl or pyrrolidinediyl groups or moieties may be one to three groups independently selected from hydroxy, hydroxy(C₁₋₄)-alkyl, (C₁₋₄)alkoxy, (C₁₋₄)alkoxy(C₁₋₄)alkyl, (C₁₋₄)alkoxy(C₁₋₄)alkoxy, (C₁₋₄)alkylsulfanyl, (C₁₋₄)-alkoxycarbonyl, (C₁₋₄)alkylcarbonyloxy, (C₁₋₄)alkylcarbonyl, (C₁₋₄)alkylsulfonyl, cyano, oxo, carboxy, carbamoyl and (C₃₋₈)cycloalkyl.

Optional substituents on amino groups or moieties can be one or two groups independently selected from (C₁₋₄)alkyl, (C₁₋₄)alkoxy(C₁₋₄)alkyl, (C₁₋₄)alkoxycarbonyl, aryl(C₁₋₄)alkoxycarbonyl and heteroaryl(C₁₋₄)alkoxycarbonyl.

Optional substituents on carbamoyl groups or moieties can be one or two groups selected from (C₁₋₄)alkyl and (C₁₋₄)alkoxy(C₁₋₄)alkyl.

Aryl or an aromatic ring is naphthyl or preferably phenyl. It can also be fused with a cycloalkyl or a heteroaromatic ring (e. g. to form a quinolyl or indolyl group).

Heteroaryl or a heteroaromatic ring is an aromatic 5- or 6-membered ring, in which 1, 2 or 3 ring atoms are hetero atoms independently selected from O, N and S, such as thiazolyl, oxazolyl or, preferably, pyridyl or pyrimidyl. It can also be fused with a cycloalkyl or an aromatic or heteroaromatic ring (e. g. to form a quinolyl or indolyl group).

A non-aromatic heterocyclyl group or moiety is a non-aromatic 5- or 6-membered cyclic structure, in which cyclic structure 1, 2 or 3 ring members are hetero ring members independently selected from the group, consisting of a nitrogen ring member, an oxygen ring member and a sulfur ring member, such as pyrrolinyl, pyrrolidyl, tetrahydrofuryl, tetrahydrothienyl, piperidyl, piperazinyl, tetrahydropyranyl or morpholinyl.

Any non-cyclic carbon containing group or moiety with more than 1 carbon atom is straight-chain or branched.

Unless defined otherwise, carbon containing groups, moieties or molecules contain 1 to 8, preferably 1 to 6, preferably 1 to 4, preferably 1 or 2, carbon atoms.

In preferred embodiments, the invention relates to a compound of the formula I, in free base form or in acid addition salt form, in which

(1) R₁ is —(CH₂)ₖN(Rₐ)R_{b}, in which
k is 0, 1 or 2;
Rₐ is hydrogen or an optionally substituted (C₁₋₈)alkyl, (C₃₋₈)cycloalkyl, (C₃₋₈)cycloalkyl-(C₁₋₄)alkyl, aryl, aryl(C₁₋₄)alkyl, heteroaryl, heteroaryl(C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydro-quinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]oxathiin-4-yl, 2,3,4,5-tetrahydro-benzo[b]oxepin-5-yl or 1,3,4,5-tetrahydro-benzo[c]oxepin-5-yl group; and
R_{b} is a (C₃₋₈)cycloalkyl group, in which
   (a) one of the carbon ring members of the (C₃₋₈)cycloalkyl moiety, which are different from the carbon ring member, to which the nitrogen atom carrying Rₐ is attached, is optionally replaced by a hetero ring member, selected from the group consisting of —O—, —S—, —S(═O)—, —S(═O)₂— and —N(R_{c})—, in which
      R_{c} is hydrogen or an optionally substituted (C₁₋₈)alkyl, (C₃₋₈)cycloalkyl, (C₃₋₈)cycloalkyl(C₁₋₄)alkyl, aryl, aryl(C₁₋₄)alkyl, heteroaryl or heteroaryl(C₁₋₄)alkyl group,
   (b) the (C₃₋₈)cycloalkyl moiety is substituted by 1 to 4 substituents, independently selected from the group consisting of halogen, cyano, oxo, hydroxy, (C₁₋₄)— alkoxy, (C₁₋₄)alkoxy(C₁₋₄)alkoxy, (C₁₋₄)alkylthio, (C₁₋₄)alkylsulfinyl, (C₁₋₄)alkylsulfonyl, (C₁₋₄)alkylcarbonyl, (C₁₋₄)alkylcarbonyloxy, (C₁₋₄)alkoxycarbonyl, (C₁₋₄)alkoxycarbonyloxy and an optionally substituted (C₁₋₈)alkyl, (C₃₋₈)cycloalkyl, (C₃₋₈)cycloalkyl-(C₁₋₄)alkyl, aryl, aryl(C₁₋₄)alkyl, heteroaryl, heteroaryl(C₁₋₄)alkyl, non-aromatic heterocyclyl, non-aromatic heterocyclyl(C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydro-quinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]oxathiin-4-yl, 2,3,4,5-tetrahydro-benzo[b]oxepin-5-yl or 1,3,4,5-tetrahydro-benzo[c]-oxepin-5-yl group, and
   (c) the (C₃₋₈)cycloalkyl moiety is optionally substituted at two adjacent carbon ring members by two substituents, which form, together with the two adjacent carbon ring members, to which they are attached, a (C₃₋₈)cycloalkyl group, in which
      (i) one of the carbon ring members of the (C₃₋₈)cycloalkyl group thus formed, which are different from the said two adjacent carbon ring members, to which the said two substituents are optionally attached, is optionally replaced by a hetero ring member, selected from the group consisting of —O—, —S—, —S(═O)—, —S(═O)₂— and —N(R_{d})—, in which
         R_{d} is hydrogen or an optionally substituted (C₁₋₈)alkyl, (C₃₋₈)cycloalkyl, (C₃₋₈)cycloalkyl(C₁₋₄)alkyl, aryl, aryl(C₁₋₄)alkyl, heteroaryl or heteroaryl(C₁₋₄)alkyl group, and
      (ii) the (C₃₋₈)cycloalkyl group thus formed is optionally substituted by 1 to 4 substituents, independently selected from the group consisting of halogen, cyano, oxo, hydroxy, (C₁₋₄)alkoxy, (C₁₋₄)alkoxy(C₁₋₄)alkoxy, (C₁₋₄)alkylthio, (C₁₋₄)alkylsulfinyl, (C₁₋₄)alkylsulfonyl, (C₁₋₄)alkylcarbonyl, (C₁₋₄)alkylcarbonyloxy, (C₁₋₄)alkoxycarbonyl, (C₁₋₄)alkoxycarbonyloxy and an optionally substituted (C₁₋₈)alkyl, (C₃₋₈)cycloalkyl, (C₃₋₈)cycloalkyl(C₁₋₄)alkyl, aryl, aryl(C₁₋₄)alkyl, heteroaryl, heteroaryl(C₁₋₄)alkyl, non-aromatic heterocyclyl, non-aromatic heterocyclyl(C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydro-quinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]-oxathiin-4-yl, 2,3,4,5-tetrahydro-benzo[b]oxepin-5-yl or 1,3,4,5-tetrahydro-benzo[c]-oxepin-5-yl group;
         preferably —(CH₂)ₖN(Rₐ)R_{b}, in which
k is 0;
Rₐ is hydrogen; and
R_{b} is a (C₃₋₈)cycloalkyl group, which (C₃₋₈)cycloalkyl group is substituted by 1 to 4 substituents, independently selected from the group consisting of halogen, cyano, oxo, hydroxy, (C₁₋₄)alkoxy, (C₁₋₄)alkoxy(C₁₋₄)alkoxy, (C₁₋₄)alkylthio, (C₁₋₄)alkylsulfinyl, (C₁₋₄)alkylsulfonyl, (C₁₋₄)alkylcarbonyl, (C₁₋₄)alkylcarbonyloxy, (C₁₋₄)alkoxycarbonyl, (C₁₋₄)alkoxycarbonyloxy and an optionally substituted (C₁₋₈)alkyl, (C₃₋₈)cycloalkyl, (C₃₋₈)cycloalkyl-(C₁₋₄)alkyl, aryl, aryl(C₁₋₄)alkyl, heteroaryl, heteroaryl(C₁₋₄)alkyl, non-aromatic heterocyclyl, non-aromatic heterocyclyl(C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydro-quinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]oxathiin-4-yl, 2,3,4,5-tetrahydro-benzo[b]oxepin-5-yl or 1,3,4,5-tetrahydro-benzo[c]oxepin-5-yl group;
   preferably —(CH₂)ₖN(Rₐ)R_{b}, in which
k is 0;
Rₐ is hydrogen; and
R_{b} is a (C₃₋₈)cycloalkyl group, which (C₃₋₈)cycloalkyl group is mono-substituted by an optionally substituted aryl or heteroaryl group;
   preferably —(CH₂)ₖN(Rₐ)R_{b}, in which
k is 0;
Rₐ is hydrogen; and
R_{b} is a (C₃₋₈)cycloalkyl group, which (C₃₋₈)cycloalkyl group is mono-substituted by an optionally substituted phenyl, pyridyl or pyrimidyl group;
   preferably —(CH₂)ₖN(Rₐ)R_{b}, in which
k is 0;
Rₐ is hydrogen; and
R_{b} is a (C₃₋₈)cycloalkyl group, which (C₃₋₈)cycloalkyl group is mono-substituted by a phenyl, pyridyl or pyrimidyl group, which phenyl, pyridyl or pyrimidyl group is mono-substituted by halogen, (C₁₋₈)alkyl or (C₁₋₆)alkoxy;
   preferably —(CH₂)ₖN(Rₐ)R_{b}, in which
k is 0;
Rₐ is hydrogen; and
R_{b} is a (C₃₋₆)cycloalkyl group, which (C₃₋₆)cycloalkyl group is mono-substituted, preferably in the 1-position, by a phenyl, pyridyl or pyrimidyl group, which phenyl, pyridyl or pyrimidyl group is mono-substituted by halogen, (C₁₋₆)alkyl or (C₁₋₆)alkoxy;
   preferably —(CH₂)ₖN(Rₐ)R_{b}, in which
k is 0;
Rₐ is hydrogen; and
R_{b} is a cyclopropyl group, which cyclopropyl group is mono-substituted, preferably in the 1-position, by a phenyl, pyridyl or pyrimidyl group, which phenyl, pyridyl or pyrimidyl group is mono-substituted by halogen, (C₁₋₆)alkyl or (C₁₋₆)alkoxy;
   (2) R₂ is hydrogen or (C₁₋₈)alkyl;
   preferably hydrogen;
   (3) R₃ is hydrogen, (C₁₋₈)alkyl or an optionally substituted (C₁₋₈)alkylOC(═O)NH, (C₃₋₈)cycloalkylOC(═O)NH, (C₃₋₈)cycloalkyl(C₁₋₄)alkylOC(═O)NH, aryl(C₁₋₄)alkylOC(═O)NH, heteroaryl(C₁₋₄)alkylOC(═O)NH, (C₁₋₄)alkylC(═O)NH, (C₃₋₈)cycloalkylC(═O)NH, arylC(═O)NH, aryl-(C₁₋₄)alkylC(═O)NH, heteroarylC(═O)NH or heteroaryl(C₁₋₄)alkylC(═O)NH group;
   preferably hydrogen;
   (4) U is a bond, CF₂, CF₂CF₂, CHF, CHFCHF, cycloprop-1,2-ylene, (C₁₋₃)alkylenoxy, (C₁₋₃)-alkylenamino, (C₁₋₈)alkylene, NRₑ or an aromatic or heteroaromatic ring, which ring is optionally substituted with halogen, (C₁₋₈)alkoxy, hydroxy or (C₁₋₈)alkyl, whereby Z and V are in ortho- or meta-position to each other, wherein
Rₑ is hydrogen, (C₁₋₈)alkyl or (C₃₋₇)cycloalkyl;
   preferably a bond or (C₁₋₃)alkylenoxy;
   (5) V is CH═CH, cycloprop-1,2-ylene, CH₂CH(OH), CH(OH)CH₂ or CR_{f}R_{f}CR_{f}R_{f}, wherein
each R_{f}, independently, is hydrogen, fluorine or (C₁₋₈)alkyl;
   preferably CH₂CH₂;
   (6) either
   V₁ is hydrogen and
   V₂ is hydroxy
   or
   V₁ and V₂ together are oxo;
   preferably V₁ is hydrogen and V₂ is hydroxy;
   (7) W is (C₁₋₈)alkylene, O, S, S(═O)₂, C(═O), C(═O)O, OC(═O), N(R_{g})C(═O), C(═O)NR_{g} or
NR_{g}, wherein
   R_{g} is hydrogen or (C₁₋₈)alkyl;
      preferably (C₁₋₈)alkylene;
      preferably (C₁₋₄)alkylene;
      preferably CH(CH₃);
      (8) X is optionally substituted (C₁₋₈)alkylene or an optionally substituted (C₃₋₈)cycloalkylene, piperidinediyl or pyrrolidinediyl group, to which group Y and C(═O)NR₂ are attached in meta-position to each other;
      preferably (C₁₋₈)alkylene;
      preferably (C₁₋₅)alkylene;
      preferably CH(CH₃) or CH₂CH(CH₃);
      (9) Y is a bond, O, S(═O)₂, S(═O)₂NRₕ, N(Rₕ)S(═O)₂, NRₕ, C(Rₕ)OH, C(═O)NRₕ, N(Rₕ)C(═O), C(═O)N(Rₕ)O or ON(Rₕ)C(═O), wherein
Rₕ is hydrogen, (C₁₋₈)alkyl or (C₃₋₈)cycloalkyl;
   preferably S(═O)₂, C(═O)NRₕ or N(Rₕ)C(═O), wherein
Rₕ is hydrogen, (C₁₋₈)alkyl or (C₃₋₈)cycloalkyl;
   preferably S(═O)₂, C(═O)NRₕ or N(Rₕ)C(═O), wherein
Rₕ is (C₁₋₄)alkyl;
   (10) Z is O, CH₂, CF₂, CHF, CH═CH, cycloprop-1,2-ylene or a bond;
   preferably O or CH₂;
   (11) n is 0 to 5;
   preferably 1 to 4;
   preferably 1 or 4;
   (12) the number of ring atoms included in the macrocyclic ring is 14, 15, 16 or 17;
   preferably 16.

The preferred embodiments (1) to (12) are preferred independently, collectively or in any combination or sub-combination.

In especially preferred embodiments, the invention relates to one or more than one of the compounds of the formula I mentioned in the Examples hereinafter, in free base form or in acid addition salt form.

In a further aspect, the invention relates to a process for the preparation of a compound of the formula I, in free base form or in acid addition salt form, comprising the steps of

a) for the preparation of a compound of the formula I, in which R₁ is N(Rₐ)R_{b}, V₁ is hydrogen and V₂ is hydroxy, reaction of a compound of the formula

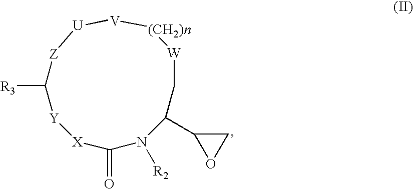

in which R₂, R₃, U, V, W, X, Y, Z and n are as defined for the formula I, with a compound of the formula HN(Rₐ)R_{b} (III), in which Rₐ and R_{b} are as defined for the formula I, or
b) cyclisation by metathesis of a suitable open chain-precursor compound, which carries, in each case, a carbon-carbon double bond at each of the two ends of the said open chain, in the presence of a catalyst, for instance a ruthenium, tungsten or molybdenum complex,
in each case optionally followed by reduction, oxidation or other functionalisation of the resulting compound and/or by cleavage of any protecting group(s) optionally present,
and of recovering the so obtainable compound of the formula I in free base form or in acid addition salt form.

The reactions can be effected according to conventional methods, for example as described in the Examples.

The working-up of the reaction mixtures and the purification of the compounds thus obtainable may be carried out in accordance with known procedures.

Acid addition salts may be prepared from free bases in known manner, and vice-versa.

Compounds of the formula I can also be prepared by further conventional processes, which processes are further aspects of the invention, e. g. as described in the Examples.

The starting materials of the formulae II and III and the open chain-precursor compounds, which are used according to process variant b), are known or may be prepared according to conventional procedures starting from known compounds, for example as described in the Examples.

Compounds of the formula I, in free base form or in pharmaceutically acceptable acid addition salt form, hereinafter often referred to as “agents of the invention”, exhibit valuable pharmacological properties, when tested in vitro or in vivo, and are, therefore, useful in medicaments.

E. g., agents of the invention are inhibitors of aspartic proteases and can be used for the treatment of a condition, disease or disorder involving processing by such enzymes. Particularly, agents of the invention inhibit beta-secretase and, thus, the generation of beta-amyloid and the subsequent aggregation into oligomers and fibrils.

The inhibiting properties of an agent of the invention towards proteases can be evaluated, e. g., in a test as described hereinafter.

### Test 1: Inhibition of Human BACE

Recombinant BACE (extracellular domain, expressed in baculovirus and purified using standard methods) at 0.1 to 10 nM concentrations is incubated with the test compound at various concentrations for 1 hour at room temperature in 10 to 100 mM acetate buffer, pH 4.5, containing 0.1% CHAPS. Synthetic fluorescence-quenched peptide substrate, derived from the sequence of APP and containing a suitable fluorophore-quencher pair, is added to a final concentration of 1 to 5 μM, and the increase in fluorescence is recorded at a suitable excitation/emission wavelength in a microplate spectro-fluorimeter for 5 to 30 minutes in 1-minute intervals. IC₅₀ values are calculated from percentage of inhibition of BACE-activity as a function of the test compound concentration.

### Test 2: Inhibition of Human BACE-2

Recombinant BACE-2 (extracellular domain, expressed in baculovirus and purified using standard methods) at 0.1 to 10 nM concentrations is incubated with the test compound at various concentrations for 1 hour at room temperature in 10 to 100 mM acetate buffer, pH 4.5, containing 0.1% CHAPS. Synthetic peptide substrate, derived from the sequence of APP and containing a suitable fluorophore-quencher pair, is added to a final concentration of 1 to 5 μM, and the increase in fluorescence is recorded at a suitable excitation/emission wavelength in a microplate spectro-fluorimeter for 5 to 30 minutes in 1-minute intervals. IC₅₀ values are calculated from percentage of inhibition of BACE-2-activity as a function of the test compound concentration.

### Test 3: Inhibition of Human Cathepsin D

Recombinant cathepsin D (expressed as procathepsin D in baculovirus, purified using standard methods and activated by incubation in sodium formate buffer pH 3.7) is incubated with the test compound at various concentrations for 1 hour at room temperature in sodium formate or sodium acetate buffer at a suitable pH within the range of pH 3.0 to 5.0. Synthetic peptide substrate Mca-Gly-Lys-Pro-lle-Leu-Phe-Phe-Arg-Leu-Lys(DNP)-D-Arg-NH₂ is added to a final concentration of 1 to 5 μM, and the increase in fluorescence is recorded at excitation of 325 nm and emission at 400 nm in a microplate spectro-fluorimeter for 5 to 30 minutes in 1-minute intervals. IC₅₀ values are calculated from the percentage of inhibition of cathepsin D-activity as a function of the test compound concentration.

### Test 4: Inhibition of Cellular Release of Amyloid Peptide 1-40

Chinese hamster ovary cells are transfected with the gene for amyloid precursor protein. The cells are plated at a density of 8000 cells/well into 96-well microtiter plates and cultivated for 24 hours in DMEM cell culture medium containing 10% FCS. The test compound is added to the cells at various concentrations, and the cells are cultivated for 24 hours in the presence of the test compound. The supernatants are collected, and the concentration of amyloid peptide 1-40 is determined using sandwich ELISA. The potency of the compound is calculated from the percentage of inhibition of amyloid peptide release as a function of the test compound concentration.

In at least one of the above-described tests, agents of the invention show activity at concentrations below 50 μM.

Specifically, the agent of the invention described in Example 1 shows an IC₅₀ value of 0.03 μM in Test 1.

Due to their inhibiting properties towards proteases, agents of the invention are useful, e. g., in the treatment or prevention of a neurological or vascular condition, disease or disorder, in which beta-amyloid generation or aggregation plays a role, such as a neurodegenerative condition, disease or disorder, e. g. Alzheimer's disease, Down's syndrome, memory impairment, cognitive impairment, dementia, amyloid neuropathies, brain inflammation, nerve trauma, brain trauma, vascular amyloidosis or cerebral hemorrhage with amyloidosis, or, based on the inhibition of BACE-2 (beta-site APP-cleaving enzyme 2) or cathepsin D, which are close homologues of the pepsin-type aspartyl proteases and beta-secretase, and the correlation of the BACE-2 or cathepsin D expression with a more tumorigenic or metastatic potential of tumor cells, in the suppression of the metastasis process associated with tumor cells.

For the above-mentioned indications, the appropriate dosage will vary depending on, e. g., the compound employed as active pharmaceutical ingredient, the host, the mode of administration, the nature and severity of the condition, disease or disorder or the effect desired. However, in general, satisfactory results in animals are indicated to be obtained at a daily dosage of from about 0.1 to about 100, preferably from about 1 to about 50, mg/kg of animal body weight. In larger mammals, for example humans, an indicated daily dosage is in the range of from about 0.5 to about 2000, preferably from about 2 to about 200, mg of an agent of the invention conveniently administered, for example, in divided doses up to four times a day or in sustained release form.

An agent of the invention may be administered by any conventional route, in particular enterally, preferably orally, e. g. in the form of a tablet or capsule, or parenterally, e. g. in the form of an injectable solution or suspension.

In accordance with the foregoing, in a further aspect, the invention relates to an agent of the invention for use as a medicament, e. g. for the treatment or prevention of a neurological or vascular condition, disease or disorder, in which beta-amyloid generation or aggregation plays a role, or for the suppression of the metastasis process associated with tumor cells.

In a further aspect, the invention relates to the use of an agent of the invention as active pharmaceutical ingredient in a medicament, e. g. for the treatment or prevention of a neurological or vascular condition, disease or disorder, in which beta-amyloid generation or aggregation plays a role, or for the suppression of the metastasis process associated with tumor cells.

In a further aspect, the invention relates to a pharmaceutical composition comprising an agent of the invention as active pharmaceutical ingredient in association with at least one pharmaceutically acceptable carrier or diluent. Such a composition may be manufactured in conventional manner, e. g. by mixing its components. Unit dosage forms contain, e. g., from about 0.1 to about 1000, preferably from about 1 to about 500, mg of an agent of the invention.

An agent of the invention can be administered as sole active pharmaceutical ingredient or as a combination with at least one other active pharmaceutical ingredient effective, e. g., in the treatment or prevention of a neurological or vascular condition, disease or disorder, in which beta-amyloid generation or aggregation plays a role, or in the suppression of the metastasis process associated with tumor cells. Such a pharmaceutical combination may be in the form of a unit dosage form, which unit dosage form comprises a predetermined quantity of each of the at least two active components in association with at least one pharmaceutically acceptable carrier or diluent. Alternatively, the pharmaceutical combination may be in the form of a package comprising the at least two active components separately, e. g. a pack or dispenser-device adapted for the concomitant or separate administration of the at least two active components, in which these active components are separately arranged. In a further aspect, the invention relates to such pharmaceutical combinations.

In a further aspect, the invention relates to the use of an agent of the invention for the manufacture of a medicament for the treatment or prevention of a neurological or vascular condition, disease or disorder, in which beta-amyloid generation or aggregation plays a role, or for the suppression of the metastasis process associated with tumor cells.

In a further aspect, the invention relates to a method for the treatment or prevention of a neurological or vascular condition, disease or disorder, in which beta-amyloid generation or aggregation plays a role, or for the suppression of the metastasis process associated with tumor cells, in a subject in need of such treatment, prevention or suppression, which method comprises administering to such subject an effective amount of an agent of the invention.

The following Examples illustrate the invention, but do not limit it.

### EXAMPLES

### Abbreviations

aq. aqueous
BF₃*Et₂O boron trifluoride-diethyl etherate
Boc tert-butoxycarbonyl
DCM dichloromethane
DIPEA diisopropylethylamine
DMSO dimethylsulfoxide
EDC.HCl 1-ethyl-3-[3-(dimethylamino)propyl]-carbodiimide hydrochloride
Et₂O diethyl ether
EtOAc ethyl acetate
EtOH ethanol
h hour(s)
HOBt hydroxybenzotriazole
MeOH methanol
min minute(s)
NH₃ 13.4 N aq. ammonia
NMR nuclear magnetic resonance spectrometry
Pd(PPh₃)₄ tetrakis(triphenylphosphine)palladium(0)
R_{f} retention factor (thin layer chromatography)
rt room temperature
THF tetrahydrofuran

### Example 1

### (3S,14R,16S)-16-{(R)-1-Hydroxy-2-[1-(3-isopropyl-phenyl)-cyclopropylamino]-ethyl}-3,4,14-trimethyl-1,4-diaza-cyclohexadecane-2,5-dione

### a) (S)—N-[(1S,3R)-1-((S)-2-Chloro-1-hydroxy-ethyl)-3-methyl-hept-6-enyl]-2-methylamino-propionamide Hydrochloride

To a solution of 814 mg (2.08 mmol) of {(S)-1-[(1S,3R)-1-((S)-2-chloro-1-hydroxy-ethyl)-3-methyl-hept-6-enylcarbamoyl]-ethyl}-methyl-carbamic acid tert-butyl ester in 4 ml of DCM are added at 0° C. 6.3 ml of 5 M HCl in Et₂O (31.3 mmol). The mixture is stirred at rt for 1.5 h and then evaporated to yield the title compound as a pale brownish powder, which is used for the next step without further purification.

¹H-NMR (400 MHz, d6-DMSO): 8.97 (br, 1H), 8.78 (br, 1H), 8.35 (d, 1H), 5.83-5.71 (m, 1H), 5.47 (d, 1H), 5.03-4.88 (m, 2H), 3.98-3.86 (m, 1H), 3.79-3.69 (m, 1H), 3.62-3.53 (m, 2H), 3.51-3.43 (m, 1H), 2.46 (s, 3H), 2.10-1.95 (m, 2H), 1.56-1.17 (m, 5H), 1.38 (d, 3H), 0.85 (d, 3H).

### b) Hept-6-enoic acid {(S)-1-[(1S,3R)-1-((S)-2-chloro-1-hydroxy-ethyl)-3-methyl-hept-6-enylcarbamoyl]-ethyl}-methyl-amide

To an ice-cold solution of 141 mg (1.1 mmol) of hept-6-enoic acid, 221 mg (1.1 mmol) of HOBt.H₂O, 230 mg (1.2 mmol) of EDC.HCl and 327 mg (1.0 mmol) of (S)—N-[(1S,3R)-1-((S)-2-chloro-1-hydroxy-ethyl)-3-methyl-hept-6-enyl]-2-methylamino-propionamide hydrochloride in 12 ml of DCM are added 0.172 ml (1.0 mmol) of DIPEA. The mixture is stirred at rt for 17 h. After cooling with ice, 10 ml of 0.5 M aq. HCl are added. The layers are separated, and the organic layer is washed with 1 M aq. potassium bicarbonate solution and water, dried over sodium sulfate and evaporated. The residue is purified by chromatography on silica gel (cyclohexane/EtOAc 70/30) to yield the title compound as a yellow solid.

¹H-NMR (400 MHz, d6-DMSO; major rotamer): 7.31 (d, 1H), 5.85-5.70 (m, 2H), 5.28 (d, 1H), 5.04-4.78 (m, 5H), 3.88-3.73 (m, 1H), 3.63-3.46 (m, 2H), 3.43-3.34 (m, 1H), 2.82 (s, 3H), 2.31 (t, 2H), 2.07-1.93 (m, 4H), 1.57-1.12 (m, 9H), 1.18 (d, 3H), 0.79 (d, 3H).

### c) (E/Z)-(3S,14R,16S)-16-((S)-2-Chloro-1-hydroxy-ethyl)-3,4,14-trimethyl-1,4-diaza-cyclohexadec-10-ene-2,5-dione

A solution of 7.05 g (17.58 mmol) of hept-6-enoic acid {(S)-1-[(1S,3R)-1-((S)-2-chloro-1-hydroxy-ethyl)-3-methyl-hept-6-enylcarbamoyl]-ethyl}-methyl-amide in 88 ml of DCM is added within 1 h to a refluxing solution of 746 mg (0.88 mmol) of [1,3-bis-(2,4,6-trimethylphenyl)-2-imidazolidinylidene)-dichloro(phenylmethylene)-(tricyclohexyl-phosphine)ruthenium] (Grubbs II catalyst) in 1.76 l of DCM. The mixture is refluxed for further 60 min, treated with 1.76 ml of butylvinylether, stirred for further 30 min, concentrated to a volume of 88 ml, poured onto a silica gel column and chromatographed (DCM to DCM/MeOH 97/3) to yield the title compound as a greyish foam.

R_{f} (cyclohexane/EtOAc 50/50): 0.15.

### d) (3S,14R,16S)-16-((S)-2-Chloro-1-hydroxy-ethyl)-3,4,14-trimethyl-1,4-diaza-cyclohexadecane-2,5-dione

A solution of 5.70 g (15.28 mmol) of (E/Z)-(3S,14R,16S)-16-((S)-2-chloro-1-hydroxy-ethyl)-3,4,14-trimethyl-1,4-diaza-cyclohexadec-10-ene-2,5-dione in 153 ml of EtOH is stirred at rt under a hydrogen atmosphere in the presence of 3.06 g of 10% Pd/carbon for 75 min. The catalyst is filtered off, the filtrate is evaporated, and the residue is purified by a first chromatography on silica gel (DCM/MeOH 98/2) and a second chromatography on silica gel (cyclohexane/EtOAc 50/50) to yield the title compound as a colorless foam.

¹H-NMR (400 MHz, d6-DMSO; major rotamer): 7.49 (t, 1H), 5.22 (t, 1H), 4.95 (q, 1H), 3.92-3.80 (m, 1H), 3.59-3.54 (m, 1H), 3.52-3.34 (m, 2H), 2.85 (s, 3H), 2.46-2.38 (m, 2H), 2.12-2.01 (m, 1H), 1.70-1.60 (m, 1H), 1.56-1.07 (m, 15H), 1.11 (d, 3H), 0.76 (d, 3H).

### e) (3S,14R,16S)-3,4,14-Trimethyl-16-(S)-oxiranyl-1,4-diaza-cyclohexadecane-2,5-dione

To a solution of 4.38 g (11.68 mmol) of (3S,14R,16S)-16-((S)-2-chloro-1-hydroxy-ethyl)-3,4,14-trimethyl-1,4-diaza-cyclohexadecane-2,5-dione in 23 ml of THF are added dropwise at 0° C. 23 ml of 1 M aq. NaOH solution (23 mmol). The mixture is stirred at 0° C. for 2 h, diluted with 230 ml of half-saturated aq. ammonium chloride solution and extracted with DCM. The combined organic layers are washed with water, dried over sodium sulfate and evaporated to yield the title compound as a colorless oil.

¹H-NMR (400 MHz, d6-DMSO; major rotamer): 7.71 (d, 1H), 4.92 (q, 1H), 3.70-3.57 (m, 1H), 2.85 (s, 3H), 2.80-2.77 (m, 1H), 2.67-2.62 (m, 1H), 2.60-2.51 (m, 2H), 2.12-2.02 (m, 1H), 1.70-1.10 (m, 17H), 1.08 (d, 3H), 0.74 (d, 3H).

### f) (3S,14R,16S)-16-{(R)-1-Hydroxy-2-[1-(3-isopropyl-phenyl)-cyclopropylamino]-ethyl}-3,4,14-trimethyl-1,4-diaza-cyclohexadecane-2,5-dione

A solution of 34 mg (0.1 mmol) of (3S,14R,16S)-3,4,14-trimethyl-16-(S)-oxiranyl-1,4-diaza-cyclohexadecane-2,5-dione in 67 mg (0.38 mmol) of 1-(3-isopropyl-phenyl)-cyclopropylamine (building block B1) is stirred at 80° C. for 4 h. The mixture is then diluted with DCM/MeOH (95/5) and purified by preparative thin layer chromatography on silica gel (DCM/MeOH 90/10) to yield the title compound as a colorless oil.

¹H-NMR (400 MHz, d6-DMSO): 7.40 (d, 1H), 7.19-7.11 (m, 2H), 7.07-6.96 (m, 2H), 4.91 (q, 1H), 4.50 (d, 1H), 3.81-3.68 (m, 1H), 2.84 (s, 3H), 2.47-2.31 (m, 4H), 2.10-1.99 (m, 2H), 1.70-1.60 (m, 2H), 1.43-1.09 (m, 16H), 1.19 (d, 6H), 1.03 (d, 3H), 0.93-0.80 (m, 4H), 0.74 (d, 3H).

### Example 1a

### (3S,14R,16S)-16-{(R)-1-Hydroxy-2-[1-(4-isopropyl-pyrid-2-yl)-cyclopropyl-amino]-ethyl}-3,4,14-trimethyl-1,4-diaza-cyclohexadecane-2,5-dione

The title compound can be prepared in a manner analogous to that described in Example 1 using in step f) building block B3 instead of building block B1.

¹H-NMR (400 MHz, d6-DMSO): 8.27 (d, 1H), 7.51 (br s, 1H), 7.47 (d, 1H), 7.00-6.96 (m, 1H), 4.95 (q, 1H), 4.68 (d, 1H), 3.84-3.74 (m, 1H), 3.39-3.33 (m, 1H), 2.89-2.85 (m, 1H), 2.84 (s, 3H), 2.58-2.53 (m, 2H), 2.47-2.32 (m, 2H), 2.10-2.01 (m, 1H), 1.71-1.61 (m, 1H), 1.51-1.11 (m, 16H), 1.22 (d, 6H), 1.04 (d, 3H), 1.00-0.90 (m, 4H), 0.76 (d, 3H).

### Example 1b

### (3S,14R,16S)-16-{(R)-2-[1-(4-tert-Butyl-pyrid-2-yl)-cyclopropylamino]-1-hydroxy-ethyl}-3,4,14-trimethyl-1,4-diaza-cyclohexadecane-2,5-dione

The title compound can be prepared in a manner analogous to that described in Example 1 using in step f) building block B4 instead of building block B1.

¹H-NMR (400 MHz, d6-DMSO): 8.29 (d, 1H), 7.59 (s, 1H), 7.06 (d, 1H), 7.00-6.85 (m, 1H), 4.89-4.75 (m, 1H), 4.26-4.15 (m, 1H), 3.87-3.74 (m, 1H), 3.46-3.34 (m, 1H), 2.87 (s, 3H), 2.67-2.56 (m, 2H), 2.43-2.14 (m, 2H), 1.70-1.15 (m, 22H), 1.32 (s, 9H), 1.04-0.94 (m, 4H), 0.81 (d, 3H).

### Example 1c

### (3S,14R,16S)-16-{(R)-1-Hydroxy-2-[1-(3-isopropoxy-phenyl)-cyclopropyl-amino]-ethyl}-3,4,14-trimethyl-1,4-diaza-cyclohexadecane-2,5-dione

The title compound can be prepared in a manner analogous to that described in Example 1 using in step f) building block B5 instead of building block B1.

¹H-NMR (400 MHz, d6-DMSO): 7.41 (d, 1H), 7.17-7.11 (m, 1H), 6.84-6.80 (m, 1H), 6.76 (t, 1H), 6.70-6.66 (m, 1H), 4.92 (q, 1H), 4.63-4.55 (m, 1H), 4.49 (d, 1H), 3.81-3.68 (m, 1H), 3.28-3.24 (m, 1H), 2.85 (s, 3H), 2.46-2.34 (m, 4H), 2.10-2.00 (m, 1H), 1.73-1.11 (m, 17H), 1.25 (d, 6H), 1.05 (d, 3H), 0.91-0.78 (m, 4H), 0.75 (d, 3H).

### Example 1d

### (3S,14R,16S)-16-{(R)-2-[1-(5-Bromo-pyrid-3-yl)-cyclopropylamino]-1-hydroxy-ethyl}-3,4,14-trimethyl-1,4-diaza-cyclohexadecane-2,5-dione

The title compound can be prepared in a manner analogous to that described in Example 1 using in step f) building block B6 instead of building block B1.

¹H-NMR (400 MHz, d6-DMSO; major rotamer): 8.49-8.44 (m, 2H), 7.93-7.89 (m, 1H), 7.43 (d, 1H), 4.92 (q, 1H), 4.55 (d, 1H), 3.81-3.77 (m, 1H), 3.29-3.24 (m, 1H), 2.85 (s, 3H), 2.64-2.52 (m, 2H), 2.45-2.30 (m, 2H), 2.11-2.03 (m, 1H), 1.71-1.60 (m, 1H), 1.51-1.09 (m, 16H), 1.05 (d, 3H), 1.03-0.94 (m, 4H), 0.75 (d, 3H).

### Example 1e

### (3S,14R,16S)-16-{(R)-2-[1-(6-tert-Butyl-pyrimid-4-yl)-cyclopropylamino]-1-hydroxy-ethyl}-3,4,14-trimethyl-1,4-diaza-cyclohexadecane-2,5-dione

The title compound can be prepared in a manner analogous to that described in Example 1 using in step f) building block B7 instead of building block B1.

¹H-NMR (400 MHz, d6-DMSO): 8.88-8.85 (m, 1H), 7.84-7.82 (m, 1H), 7.49 (d, 1H), 4.95 (q, 1H), 4.76 (d, 1H), 3.84-3.76 (m, 1H), 3.41-3.35 (m, 1H), 2.84 (s, 3H), 2.59-2.53 (m, 2H), 2.48-2.37 (m, 2H), 2.10-2.00 (m, 1H), 1.69-1.61 (m, 1H), 1.47-1.06 (m, 20H), 1.32 (s, 9H), 1.04 (d, 3H), 0.76 (d, 3H).

### Example 2

### (5S,8S,10R)-8-{(R)-1-Hydroxy-2-[1-(3-isopropyl-phenyl)-cyclopropylamino]-ethyl}-4,5,10-trimethyl-1-oxa-4,7-diaza-cyclohexadecane-3,6-dione

The title compound can be prepared in a manner analogous to that described in Example 1 using in step b) but-3-enyloxy-acetic acid instead of hept-6-enoic acid.

¹H-NMR (400 MHz, d6-DMSO): 7.49 (d, 1H), 7.18 (t, 1H), 7.13 (br s, 1H), 7.06-7.00 (m, 2H), 4.79 (q, 1H), 4.52 (d, 1H), 4.34 (d, 1H), 3.87-3.78 (m, 1H), 3.74 (d, 1H), 3.58-3.49 (m, 1H), 3.41-3.34 (m, 1H), 2.85 (s, 3H), 2.48-2.31 (m, 4H), 1.61-1.48 (m, 2H), 1.45-1.07 (m, 12H), 1.20 (d, 6H), 1.05 (d, 3H), 0.95-0.78 (m, 4H), 0.76 (d, 3H).

### Example 2a

### (5S,8S,10R)-8-{(R)-2-[1-(3-tert-Butyl-phenyl)-cyclopropylamino]-1-hydroxy-ethyl}-4,5,10-trimethyl-1-oxa-4,7-diaza-cyclohexadecane-3,6-dione

The title compound can be prepared in a manner analogous to that described in Example 2 using in step f) building block B2 instead of building block B1.

¹H-NMR (400 MHz, d6-DMSO): 7.47 (d, 1H), 7.30 (s, 1H), 7.17-7.13 (m, 2H), 7.00-6.96 (m, 1H), 4.77 (q, 1H), 4.52 (d, 1H), 4.32 (d, 1H), 3.84-3.76 (m, 1H), 3.72 (d, 1H), 3.55-3.49 (m, 1H), 3.39-3.33 (m, 1H), 3.29-3.26 (m, 1H), 2.84 (s, 3H), 2.47-2.31 (m, 2H), 1.59-1.46 (m, 2H), 1.42-1.07 (m, 12H), 1.27 (s, 9H), 1.03 (d, 3H), 0.94-0.80 (m, 4H), 0.74 (d, 3H).

### Example 2b

### (5S,8S,10R)-8-{(R)-1-Hydroxy-2-[1-(4-isopropyl-pyrid-2-yl)-cyclopropyl-amino]-ethyl}-4,5,10-trimethyl-1-oxa-4,7-diaza-cyclohexadecane-3,6-dione

The title compound can be prepared in a manner analogous to that described in Example 2 using in step f) building block B3 instead of building block B1.

¹H-NMR (400 MHz, d6-DMSO): 8.27 (d, 1H), 7.55 (d, 1H), 7.51 (s, 1H), 7.00-6.97 (m, 1H), 4.80 (q, 1H), 4.69 (d, 1H), 4.34 (d, 1H), 4.11-4.06 (m, 1H), 3.89-3.81 (m, 1H), 3.74 (d, 1H), 3.58-3.51 (m, 1H), 3.41-3.34 (m, 2H), 2.91-2.86 (m, 1H), 2.85 (s, 3H), 2.59-2.53 (m, 1H), 2.48-2.43 (m, 1H), 1.61-1.48 (m, 2H), 1.46-1.08 (m, 11H), 1.22 (d, 6H), 1.05 (d, 3H), 1.00-0.85 (m, 4H), 0.77 (d, 3H).

### Example 3

### (8S,11S,13R)-11-{(R)-1-Hydroxy-2-[1-(3-isopropyl-phenyl)-cyclopropylamino]-ethyl}-7,8,13-trimethyl-1-oxa-7,10-diaza-cyclohexadecane-6,9-dione

The title compound can be prepared in a manner analogous to that described in Example 1 using as starting material in step a) the product obtainable, in a manner analogous to that described in step b) of Example 1, from the reaction of building block A1 with Boc-N-methyl-(L)-alanine and using in step b) but-3-enoic acid instead of hept-6-enoic acid.

¹H-NMR (400 MHz, d6-DMSO; major rotamer): 7.66 (d, 1H), 7.17 (t, 1H), 7.13 (br s, 1H), 7.10-6.99 (m, 2H), 4.59 (t, 1H), 4.47-4.42 (m, 1H), 3.74-3.66 (m, 1H), 3.26-3.16 (m, 2H), 2.86 (s, 3H), 2.49-2.30 (m, 2H), 2.11-1.99 (m, 1H), 1.76-1.04 (m, 18H), 1.19 (d, 6H), 0.93-0.80 (m, 4H), 0.74 (t, 3H).

### Example 4

### (3S,6S,8R)-6-{(R)-2-[1-(3-tert-Butyl-phenyl)-cyclopropylamino]-1-hydroxy-ethyl}-3,8-dimethyl-1,1-dioxo-1lambda*6*-thia-5-aza-cyclohexadecan-4-one

The title compound can be prepared in a manner analogous to that described in Example 1 omitting steps a) and b), using as starting material in step c) the product obtainable, in a manner analogous to that described in step b) of Example 1, from the reaction of (2S,3S,5R)-3-amino-1-chloro-5-methyl-non-8-en-2-ol hydrochloride with (S)-3-(hex-5-ene-1-sulfonyl)-2-methyl-propionic acid and using in step f) building block B2 instead of building block B1.

¹H-NMR (400 MHz, d6-DMSO): 7.77 (d, 1H), 7.33 (s, 1H), 7.18-7.15 (m, 1H), 7.02-6.98 (m, 1H), 4.59 (d, 1H), 3.77-3.68 (m, 1H), 3.29-3.09 (m, 2H), 3.05-2.98 (m, 1H), 2.90-2.77 (m, 2H), 2.47-2.32 (m, 2H), 1.64-1.54 (m, 2H), 1.45-1.11 (m, 18H), 1.28 (s, 9H), 1.07 (d, 3H), 0.92-0.80 (m, 4H), 0.75 (d, 3H).

### Building block A1

### (2S,3S,5R)-8-Allyloxy-3-amino-1-chloro-5-methyl-octan-2-ol Hydrochloride

The title compound can be prepared in a manner analogous to known procedures, starting from (2S,4R)-2-tert-butoxycarbonylamino-4-methyl-oct-7-enoic acid methyl ester, via the following reaction sequence:

Ozonolysis of the terminal double bond, reduction of the resulting aldehyde to the primary alcohol, cleavage of the Boc protecting group, re-protection of the amino group with the phenylfluorenyl protecting group, alkylation of the primary alcohol with allylbromide, cleavage of the phenylfluorenyl protecting group and re-protection of the amino group with the Boc protecting group yields the (2S,4R)-7-allyloxy-2-tert-butoxycarbonylamino-4-methyl-heptanoic acid methyl ester [¹H-NMR (400 MHz, d6-DMSO): 7.79 (br s, 3H), 5.95-5.80 (m, 2H), 5.24-5.18 (m, 1H), 5.13-5.09 (m, 1H), 3.92-3.88 (m, 3H), 3.86-3.62 (m, 1H), 3.57-3.52 (m, 1H), 3.35 (t, 2H), 3.29-3.23 (m, 1H), 1.60-1.44 (m, 4H), 1.31-1.16 (m, 3H), 0.84 (d, 3H)], which is further transformed by chloroketone formation, reduction to the chlorohydrine and cleavage of the Boc protecting group to yield the title compound.

¹H-NMR (400 MHz, d6-DMSO): 7.21 (d, 1H), 5.91-5.81 (m, 1H), 5.25-5.18 (m, 1H), 5.13-5.09 (m, 1H), 4.05-3.97 (m, 1H), 3.91-3.88 (m, 2H), 3.61 (s, 3H), 3.34 (t, 2H), 1.66-1.42 (m, 4H), 1.40-1.12 (m, 3H), 1.38 (s, 9H), 0.83 (d, 3H).

### Building block B1

### 1-(3-Isopropyl-phenyl)-cyclopropylamine

### a) 3-Isopropyl-benzonitrile

To a solution of 200 g (954 mmol) of 1-bromo-3-isopropyl-benzene in 1 l of 1-methyl-2-pyrrolidone are added under a nitrogen atmosphere 114 g (954 mmol) of zinc cyanide and 28.7 g (24.8 mmol) of Pd(PPh₃)₄. The mixture is heated to 125° C., stirred at this temperature for 150 min, then cooled to rt and filtered through Hyflo Super Gel. The filtrate is diluted with water and EtOAc. The organic layer is washed with water, 1 N aq. HCl and brine, dried over sodium sulfate and concentrated. The residue is purified by column chromatography (DCM/hexanes 1/3) to yield the title compound.

¹H-NMR (400 MHz, CDCl₃): 7.57 (s, 1H), 7.53-7.48 (m, 2H), 7.43 (t, 1H), 3.01-2.92 (m, 1H), 1.29 (d, 6H).

### b) 1-(3-Isopropyl-phenyl)-cyclopropylamine

To a solution of 42 g (286 mmol) of 3-isopropyl-benzonitrile in 670 ml of Et₂O are added under an argon atmosphere 90.4 g (315 mmol) of titanium(IV)-isopropoxide. The mixture is cooled to −70° C., and 210 ml (630 mmol) of ethyl magnesium bromide (3 M in Et₂O) are added within 60 min. The mixture is warmed to 10° C., and 169 g (573 mmol) of BF₃*Et₂O (48%) are added at this temperature. After stirring for 1 h, the mixture is quenched with 400 ml of 1 N aq. HCl, basified to pH 10 using aq. 2 N NaOH solution and filtered through Hyflo Super Gel. The organic layer is dried over sodium sulfate and concentrated. The residue is purified by column chromatography using DCM/MeOH (19/1) to yield the title compound.

¹H-NMR (400 MHz, CDCl₃): 7.32-7.28 (t, 1H), 7.23 (s, 1H), 7.19-7.10 (m, 2H), 3.01-2.90 (m, 1H), 1.96 (s, 2H), 1.33 (d, 6H), 1.12-1.09 (m, 2H), 1.09-1.02 (m, 2H).

The building blocks B2 to B7 can be prepared in a manner analogous to that described for building block B1 via the corresponding nitriles, which are commercially available or can be prepared in a manner analogous to known procedures.

### Building block B2

### 1-(3-tert-Butyl-phenyl)-cyclopropylamine

¹H-NMR (400 MHz, d6-DMSO): 7.40-7.37 (m, 1H), 7.28-7.26 (m, 2H), 7.16-7.12 (m, 1H), 1.35 (s, 9H), 1.10-1.06 (m, 2H), 1.02-0.98 (m, 2H).

### Building block B3

### 1-(4-Isopropyl-pyrid-2-yl)-cyclopropylamine

¹H-NMR (400 MHz, d6-DMSO): 8.23 (d, 1H), 7.61 (d, 1H), 6.95 (dd, 1H), 2.19-2.80 (m, 1H), 1.21 (d, 6H), 1.17 (q, 2H), 0.91 (q, 2H).

### Building block B4

### 1-(4-tert-Butyl-pyrid-2-yl)-cyclopropylamine

¹H-NMR (400 MHz, d6-DMSO): 8.26 (d, 1H), 7.77 (d, 1H), 7.08 (dd, 1H), 1.29 (s, 9H), 1.21-1.16 (m, 2H), 0.95-0.91 (m, 2H).

### Building block B5

### 1-(3-Isopropoxy-phenyl)-cyclopropylamine

¹H-NMR (400 MHz, d6-DMSO): 7.11 (t, 2H), 6.86 (t, 1H), 6.73-6.71 (m, 1H), 6.66-6.63 (m, 1H), 4.62-4.53 (m, 1H), 2.24 (br s, 2H), 1.24 (d, 6H), 0.93-0.89 (m, 2H), 0.87-0.84 (m, 2H).

### Building block B6

### 1-(5-Bromo-pyrid-3-yl)-cyclopropylamine

¹H-NMR (400 MHz, d6-DMSO): 8.42 (t, 2H), 7.94 (t, 1H), 1.01 (d, 4H).

### Building block B7

### 1-(6-tert-Butyl-pyrimid-4-yl)-cyclopropylamine

R_{f} (DCM/MeOH/NH₃ 90/9/1): 0.45.

1. A compound of the formula 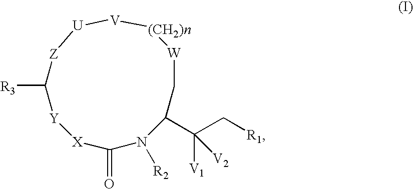
in which
R₁ is —(CH₂)ₖN(Rₐ)R_{b}, in which
k is 0, 1 or 2;
Rₐ is hydrogen or an optionally substituted (C₁₋₈)alkyl, (C₃₋₈)cycloalkyl, (C₃₋₈)cycloalkyl-(C₁₋₄)alkyl, aryl, aryl(C₁₋₄)alkyl, heteroaryl, heteroaryl(C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydro-quinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]oxathiin-4-yl, 2,3,4,5-tetrahydro-benzo[b]oxepin-5-yl or 1,3,4,5-tetrahydro-benzo[c]oxepin-5-yl group; and
R_{b} is a (C₃₋₈)cycloalkyl group, in which
(a) one of the carbon ring members of the (C₃₋₈)cycloalkyl moiety, which are different from the carbon ring member, to which the nitrogen atom carrying Rₐ is attached, is optionally replaced by a hetero ring member, selected from the group consisting of —O—, —S—, —S(═O)—, —S(═O)₂— and —N(R_{c})—, in which
R_{c} is hydrogen or an optionally substituted (C₁₋₈)alkyl, (C₃₋₈)cycloalkyl, (C₃₋₈)cycloalkyl(C₁₋₄)alkyl, aryl, aryl(C₁₋₄)alkyl, heteroaryl or heteroaryl(C₁₋₄)alkyl group,
(b) the (C₃₋₈)cycloalkyl moiety is substituted by 1 to 4 substituents, independently selected from the group consisting of halogen, cyano, oxo, hydroxy, (C₁₋₄)alkoxy, (C₁₋₄)alkoxy(C₁₋₄)alkoxy, (C₁₋₄)alkylthio, (C₁₋₄)alkylsulfinyl, (C₁₋₄)alkylsulfonyl, (C₁₋₄)alkylcarbonyl, (C₁₋₄)alkylcarbonyloxy, (C₁₋₄)alkoxycarbonyl, (C₁₋₄)alkoxycarbonyloxy and an optionally substituted (C₁₋₈)alkyl, (C₃₋₈)cycloalkyl, (C₃₋₈)cycloalkyl-(C₁₋₄)alkyl, aryl, aryl(C₁₋₄)alkyl, heteroaryl, heteroaryl(C₁₋₄)alkyl, non-aromatic heterocyclyl, non-aromatic heterocyclyl(C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydro-quinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]oxathiin-4-yl, 2,3,4,5-tetrahydro-benzo[b]oxepin-5-yl or 1,3,4,5-tetrahydro-benzo[c]oxepin-5-yl group, and
(c) the (C₃₋₈)cycloalkyl moiety is optionally substituted at two adjacent carbon ring members by two substituents, which form, together with the two adjacent carbon ring members, to which they are attached, a (C₃₋₈)cycloalkyl group, in which
(i) one of the carbon ring members of the (C₃₋₈)cycloalkyl group thus formed, which are different from the said two adjacent carbon ring members, to which the said two substituents are optionally attached, is optionally replaced by a hetero ring member, selected from the group consisting of —O—, —S—, —S(═O)—, —S(═O)₂— and —N(R_{d})—, in which
R_{d} is hydrogen or an optionally substituted (C₁₋₈)alkyl, (C₃₋₈)cycloalkyl, (C₃₋₈)cycloalkyl(C₁₋₄)alkyl, aryl, aryl(C₁₋₄)alkyl, heteroaryl or heteroaryl(C₁₋₄)alkyl group, and
(ii) the (C₃₋₈)cycloalkyl group thus formed is optionally substituted by 1 to 4 substituents, independently selected from the group consisting of halogen, cyano, oxo, hydroxy, (C₁₋₄)alkoxy, (C₁₋₄)alkoxy(C₁₋₄)alkoxy, (C₁₋₄)alkylthio, (C₁₋₄)alkylsulfinyl, (C₁₋₄)alkylsulfonyl, (C₁₋₄)alkylcarbonyl, (C₁₋₄)alkylcarbonyloxy, (C₁₋₄)alkoxycarbonyl, (C₁₋₄)alkoxycarbonyloxy and an optionally substituted (C₁₋₈)alkyl, (C₃₋₈)cycloalkyl, (C₃₋₈)cycloalkyl(C₁₋₄)alkyl, aryl, aryl(C₁₋₄)alkyl, heteroaryl, heteroaryl(C₁₋₄)alkyl, non-aromatic heterocyclyl, non-aromatic heterocyclyl(C₁₋₁₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydro-quinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]oxathiin-4-yl, 2,3,4,5-tetrahydro-benzo[b]oxepin-5-yl or 1,3,4,5-tetrahydro-benzo[c]oxepin-5-yl group;
R₂ is hydrogen or (C₁₋₈)alkyl;
R₃ is hydrogen, (C₁₋₈)alkyl or an optionally substituted (C₁₋₈)alkylOC(═O)NH, (C₃₋₈)cycloalkylOC(═O)NH, (C₃₋₈)cycloalkyl(C₁₋₄)alkylOC(═O)NH, aryl(C₁₋₄)alkylOC(═O)NH, heteroaryl(C₁₋₄)alkylOC(═O)NH, (C₁₋₄)alkylC(═O)NH, (C₃₋₈)cycloalkylC(═O)NH, arylC(═O)NH, aryl(C₁₋₄)alkylC(═O)NH, heteroarylC(═O)NH or heteroaryl(C₁₋₄)alkylC(═O)NH group;
U is a bond, CF₂, CF₂CF₂, CHF, CHFCHF, cycloprop-1,2-ylene, (C₁₋₃)alkylenoxy, (C₁₋₃)alkylenamino, (C₁₋₈)alkylene, NRₑ or an aromatic or heteroaromatic ring, which ring is optionally substituted with halogen, (C₁₋₈)alkoxy, hydroxy or (C₁₋₈)alkyl, whereby Z and V are in ortho- or meta-position to each other, wherein
Rₑ is hydrogen, (C₁₋₈)alkyl or (C₃₋₇)cycloalkyl;
V is CH═CH, cycloprop-1,2-ylene, CH₂CH(OH), CH(OH)CH₂ or CR_{f}R_{f}CR_{f}R_{f}, wherein
each R_{f}, independently, is hydrogen, fluorine or (C₁₋₈)alkyl;
either
V, is hydrogen and
V₂ is hydroxy
or
V₁ and V₂ together are oxo;
W is (C₁₋₈)alkylene, O, S, S(═O)₂, C(═O), C(═O)O, OC(═O), N(R_{g})C(═O), C(═O)NR_{g} or NR_{g}, wherein
R_{g} is hydrogen or (C₁₋₈)alkyl;
X is optionally substituted (C₁₋₈)alkylene or an optionally substituted (C₃₋₈)cycloalkylene, piperidinediyl or pyrrolidinediyl group, to which group Y and C(═O)NR₂ are attached in meta-position to each other;
Y is a bond, O, S(═O)₂, S(═O)₂NRₕ, N(Rₕ)S(═O)₂, NRₕ, C(Rₕ)OH, C(═O)NRₕ, N(Rₕ)C(═O), C(═O)N(Rₕ)O or ON(Rₕ)C(═O), wherein
Rₕ is hydrogen, (C₁₋₈)alkyl or (C₃₋₈)cycloalkyl;
Z is O, CH₂, CF₂, CHF, CH═CH, cycloprop-1,2-ylene or a bond; and
n is 0 to 5,
the number of ring atoms included in the macrocyclic ring being 14, 15, 16 or 17,
in free base form or in acid addition salt form.

2. A process for the preparation of a compound as defined in claim 1 of the formula I, in free base form or in acid addition salt form, comprising the steps of
a) for the preparation of a compound of the formula I, in which R₁ is N(Rₐ)R_{b}, V₁ is hydrogen and V₂ is hydroxy, reaction of a compound of the formula 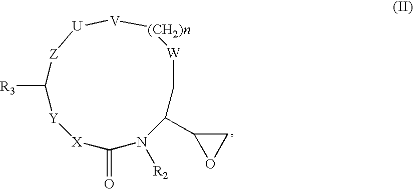
in which R₂, R₃, U, V, W, X, Y, Z and n are as defined for the formula I, with a compound of the formula HN(Rₐ)R_{b} (III), in which Rₐ and R_{b} are as defined for the formula I, or
b) cyclisation by metathesis of a suitable open chain-precursor compound, which carries, in each case, a carbon-carbon double bond at each of the two ends of the said open chain, in the presence of a catalyst, for instance a ruthenium, tungsten or molybdenum complex,
in each case optionally followed by reduction, oxidation or other functionalisation of the resulting compound and/or by cleavage of any protecting group(s) optionally present,
and of recovering the so obtainable compound of the formula I in free base form or in acid addition salt form.

3-4. (canceled)

5. A pharmaceutical composition, comprising:
the compound as defined in claim 1 of the formula I, in free base form or in pharmaceutically acceptable acid addition salt form, as active ingredient and
a pharmaceutical carrier or diluent.

6-7. (canceled)

8. A method for the treatment of neurological or vascular disorders related to beta-amyloid generation and/or aggregation in a subject in need of such treatment, comprising:
administering to such subject a therapeutically effective amount of the compound as defined in claim 1 of the formula I, in free base form or in pharmaceutically acceptable acid addition salt form.

9. A combination, comprising:
a therapeutically effective amount of the compound as defined in claim 1 of the formula I, in free base form or in pharmaceutically acceptable acid addition salt form, and
a second drug substance, for simultaneous or sequential administration.
